(19) United States
(12) Patent Application Publication
(10) Pub. No.: US 2025/0288742 A1
(43) Pub. Date: Sep. 18, 2025

(54) WEARABLE DEVICE FOR RECOGNITION AND RESPONSE TO AN OVERDOSE

(71) Applicants: The Trustees of the University of Pennsylvania, Philadelphia, PA (US); Drexel University, Philadelphia, PA (US)

(72) Inventors: Jacob S. Brenner, Princeton Junction, NJ (US); Anush Lingamoorthy, Philadelphia, PA (US); David Gordon, Downingtown, PA (US); Ethan Cornelius Donlon, Concord, NH (US); Cameron Baston, Philadelphia, PA (US); Khang Lam, Philadelphia, PA (US)

(21) Appl. No.: 18/860,489

(22) PCT Filed: Apr. 26, 2023
(86) PCT No.: PCT/US2023/019903

§ 371 Date: Oct. 25, 2024

Related U.S. Application Data
(60) Provisional application No. 63/335,657, filed on Apr. 27, 2022.

(51) Int. Cl. A61M 5/20; A61K 9/00; A61K 31/4178; A61K 31/485

(52) U.S. Cl. CPC A61M 5/2033; A61K 9/0019; A61K 31/4178; A61K 31/485; A61M 2005/206; A61M 2005/208; A61M 2205/18; A61M 2205/276; A61M 2205/3303; A61M 2209/088; A61M 2230/20; A61M 2230/63

(57) ABSTRACT

A wearable device is provided for recognition and response to an overdose, such as an opioid overdose. The device includes an oximetry sensor for measuring peripheral oxygen saturation (SpO₂) of a wearer of the wearable device and a motion sensor for determining whether the wearer of the wearable device is moving or motionless. The wearable device further includes an injector including a supply of an antidote and a needle for intramuscular delivery of the antidote to the wearer of the wearable device. The wearable device may also include a housing in which the oximetry sensor, the motion sensor, and the supply of the antidote is contained and in which the needle is initially contained in a retracted condition. The wearable device may further include a strap extending from the housing and sized to secure the housing on an upper arm of the wearer adjacent the shoulder of the wearer.

### BACKGROUND

Embodiments disclosed herein are directed to a wearable device that is able to sense a drug overdose by the wearer of the device and automatically administer an antidote and/or contact responders.

Drug overdoses, such as opioid overdoses, render the drug user in a condition with altered metal status where they are unable to respond. Often, these individuals may be unconscious. Therefore, they require the intervention of another individual to attempt to reverse the overdose. In particular, the current standard of care for opioid overdose is the administration of naloxone, an opioid-receptor antagonist first synthesized in 1961, that prevents opioids from binding to the receptor and counteracts the respiratory depression induced by opioids. Naloxone is approved by the FDA for intranasal, intramuscular, intravenous, and subcutaneous delivery and is known to reverse an opioid overdose within minutes.

Accordingly, overdose prevention has conventionally focused primarily on equipping first responders, community members, family members, etc., with naloxone. While this approach has effectively reversed countless overdoses, fatal opioid overdoses continue to occur when a bystander is not otherwise present to administer naloxone-in fact, it is estimated that 52% of opioid overdoses occur simply because someone was using opioids alone. Further, every minute of delay waiting for a bystander to administer naloxone results in brain damage, leading to permanent decreased cognitive performance.

Opioid overdoses have skyrocketed in the era of COVID-19. The intertwined burden of the ongoing opioid crisis and the global COVID-19 pandemic has revealed an urgent need for novel solutions to reverse opioid overdoses such as when individuals use opioids alone.

Though naloxone is safe and effective at reversing opioid overdose, the major limitation is the requirement that the overdose be identified and reversed by another person. And, although naloxone is accessible via intranasal naloxone spray, Evzio injector, and vials—both through pharmacies and provided upon discharge from an emergency department—these formulations all require a bystander to assess the overdose and administer the naloxone.

By way of example, studies have found 51.8% of fatal overdoses in Philadelphia were not witnessed by another individual, and 27.4% of fatal overdoses were witnessed by a bystander who failed to recognize the symptoms of an opioid overdose. Thus, there is a tremendous, demonstrated need for overdose prevention modalities that do not solely rely on a bystander's presence or judgment. Neither of the existing, FDA-approved naloxone formulations, the intranasal Narcan spray nor the Evzio auto-injector, can prevent these deaths because both require a bystander to administer the naloxone.

### SUMMARY OF THE INVENTION

In one aspect, a wearable device is provided for recognition and response to an overdose, such as an opioid overdose. The device includes an oximetry sensor for measuring peripheral oxygen saturation (SpO₂) of a wearer of the wearable device and a motion sensor for determining whether the wearer of the wearable device is moving or motionless.

According to another aspect, the wearable device further includes an injector including a supply of an antidote and a needle for intramuscular delivery of the antidote to the wearer of the wearable device. The wearable device may also include a housing in which the oximetry sensor, the motion sensor, and the supply of the antidote is contained and in which the needle is initially contained in a retracted condition. The wearable device may further include a strap extending from the housing and sized to secure the housing on an upper arm of the wearer adjacent the shoulder of the wearer.

According to yet another aspect, the wearable device may further include control electronics housed within the housing and configured to receive and analyze readings of the oximetry sensor and the motion sensor and to detect a suspected overdose from the readings. For instance, the control electronics may be configured to determine a suspected overdose when peripheral oxygen saturation (SpO₂) measured by the oximetry sensor falls below a predetermined oxygen saturation threshold level and the motion sensor indicates that the wearer of the wearable device is motionless.

According to other aspects, the wearable device may be configured to titrate delivery of the antidote such that only a portion of the antidote carried by the wearable device is injected followed by analysis of additional sensor readings to determine whether or not a further injection of an additional portion of the antidote is required. Still further, the antidote may be naloxone or a mixture of naloxone an antiemetic.

### BRIEF DESCRIPTION OF THE DRAWINGS

FIG. 1A-1C are views showing use of a wearable smart device according to an embodiment.

FIG. 2A is a perspective view of a wearable smart device according to an embodiment and FIG. 2B is an exploded view of the device of FIG. 2A.

FIG. 3 is a block diagram of the operation of a wearable smart device according to an embodiment.

FIG. 4 is an exploded view of an alternate embodiment of a wearable smart device.

FIG. 5 is a perspective view of another embodiment of a wearable smart device.

FIG. 6 is a view of the device of FIG. 5 as worn on the upper arm of a wearer according to an embodiment.

FIG. 7 shows a possible location for the device on an upper thigh of a wearer according to an embodiment.

FIG. 8 is perspective view of an adhesive pad for mounting a wearable smart device according to an embodiment.

FIG. 9 is a perspective view of a wearable smart device having a strap according to an embodiment.

FIG. 10 is a perspective view of an alternate strap for a wearable smart device according to an embodiment.

FIG. 11 is an exploded view of yet a further alternate embodiment of a wearable smart device.

FIGS. 12 and 13 are perspective views of the injector of FIG. 11.

FIG. 14 is a flow chart of sensor operation according to an embodiment.

FIG. 15 is a graph of showing light attenuation by tissue components of the wearer over time.

FIG. 16 is a graph of showing AC/DC components calculated from the Analogue to Digital Converter (ADC) count of both Red and IR signals according to an embodiment.

FIG. 17 is a flow chart of a calibration operation of the sensors according to an embodiment.

FIG. 18 are images showing different Pantone skin tones.

FIG. 19 is an image showing a top plan view of the electronics of an embodiment of the wearable device with housing removed.

FIG. 20 is an image showing a bottom plan view of the electronics of an embodiment of the wearable device with housing removed.

FIG. 21 is an image showing a vial for use in a physiology-based titration mechanism according to an embodiment.

FIG. 22 is an image showing a physiology-based titration mechanism according to an embodiment.

### DETAILED DESCRIPTION OF THE INVENTION

### Definitions

The following definitions are provided. It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

As used herein, a “subject,” “patient,” or “wearer” is a human.

Unless defined otherwise in this specification, technical and scientific terms used herein have the same meaning as commonly understood by one of ordinary skill in the art and by reference to published texts, which provide one skilled in the art with a general guide to many of the terms used in the present application.

### Wearable Smart Device

Various embodiments of wearable smart devices are disclosed herein and each provide much-needed utility by removing the need for a bystander to administer naloxone or like antidote in the event of an opioid or like overdose. According to at least some embodiments, a wearable device actively senses for an overdose of the wearer of the device and, if an overdose event is detected, automatically injects an antidote, such as naloxone, into the wearer and/or contacts another for assistance by transmitting a voice or text message or alert, such as via a wireless transmission. Thus, upon sensing an overdose, the wearable smart device may automatically actuate any or all of multiple different programs that may be pre-chosen by the user/wearer of the device.

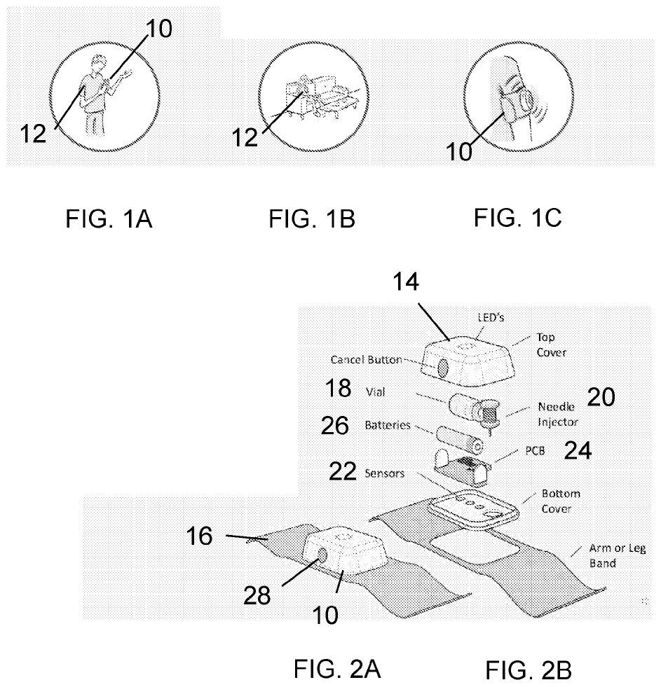

By way of a first example, FIGS. 1A-1C disclose an intended use and operation of a wearable smart device 10 by a wearer 12. In FIG. 1A, the device 10 is secured to the wearer 12 and begins to sense conditions of the patient, for instance blood oxygenation, as well as movements of the wearer at a time, for instance, before, during and after opioid use. In FIG. 1B, if the device 10 recognizes a life-threatening overdose event (i.e., such as by sensing blood oxygenation below a preset threshold and lack of movement by the wearer), the device 10 may initially activate an alarm (audible, vibratory, or the like) to notify the wearer or any bystander that may be nearby. If the user or bystander, if any, does not respond to the alarm within a preset amount of time, the device 10 may be set to automatically: alert first responders via a call, text message, etc.; alert nearby community members, family, or friends via a transmitted message to a mobile app or the like; and/or inject naloxone or like antidote contained within the device 10. See FIG. 1C.

Accordingly, the wearable smart device 10 can provide reliable and timely treatment for unintentional opioid overdose and can thereby help to reduce death and severe disability. The wearable device may be designed as a single use device that users may be able to preset such that the device notifies proper authorities, or a designated family member or friend should an overdose condition be detected.

Structurally, the device 10 can include: i) an auto-injector mechanism; ii) a sensing system; and iii) a stabilization system.

For instance, as shown in FIGS. 2A and 2B, the device 10 may include a housing 14 for the auto-injector mechanism and the sensing system and may include an arm, shoulder, or leg strap or band 16 or other stabilization system. The auto-injector mechanism may include a vessel 18 containing a supply of antidote and a needle injector 20 having a needle for being advanced intramuscularly in a wearer upon detection of an overdose. The sensing system may include sensors 22 for sensing blood oxygenation and motion of the wearer. A printed circuit board 24 may be provided with electronic components for receiving and interpreting the information detected by the sensors 22. The electronic components may include a transmitter for transmitting signals or messages and may include connection to a speaker or the like. The device 10 may be battery-powered via a battery 26 contained within the housing 14 and may include indicating LEDs and an override button or switch 28. The indicating LEDs may indicate the condition of the device 10 such as battery level and operational status (i.e., the device is powered and functional to sense for an overdose event, or the device has sensed an overdose event).

The control electronics of the sensing system of the device for sensing an overdose may use an algorithm to validate sensor readings from FDA-approved sensor technology to classify patient status. A parameter invariant algorithm approach may be used. For instance, the sensing system may use a parameter invariant (PAIN) algorithm to validate sensor readings to properly classify patient status.

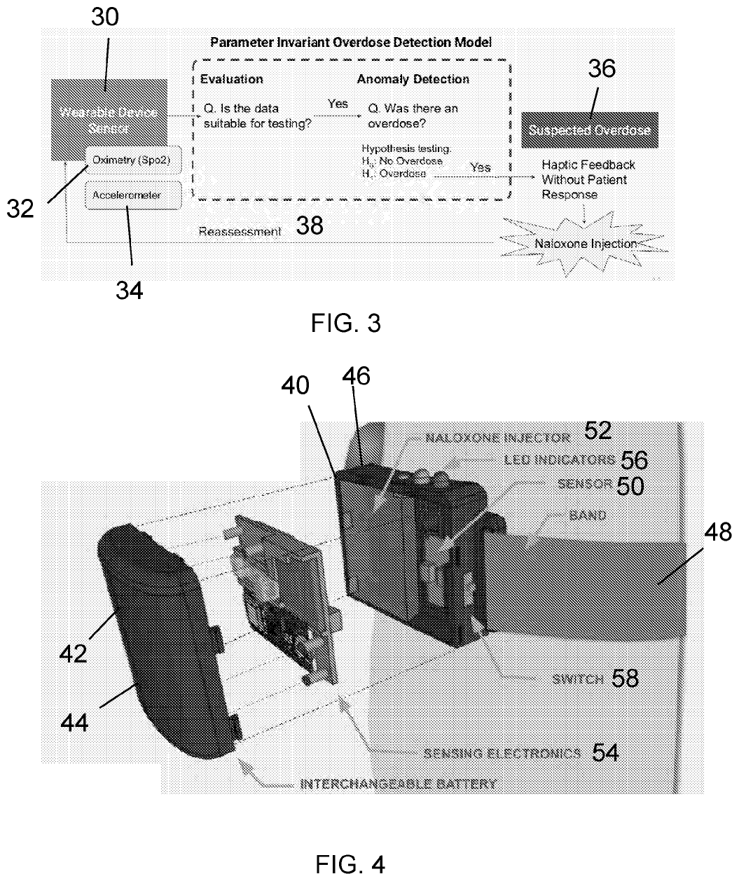

By way of example, as shown in FIG. 3, the sensing system 30 of the wearable device 10 may include an oximetry (SpO₂) sensor 32 and a motion sensor, such as an accelerometer 34. If the sensed data indicates that the wearer is motionless and the oxygen saturation level has fallen below a preset threshold for a preset amount of time, then the control electronics of the sensing system 30 may recognize a suspected overdose condition 36. An alarm or the like is sounded, and if there is no response to the alarm, such as the depression of the override button 28 or movement of the wearer, via a reassessment 38, then the auto-injector mechanism will be activated and/or the transmission of messages or alerts to responders or other community members will occur. As desired, the device may be preset to take all of these actions, or just selected ones of the actions.

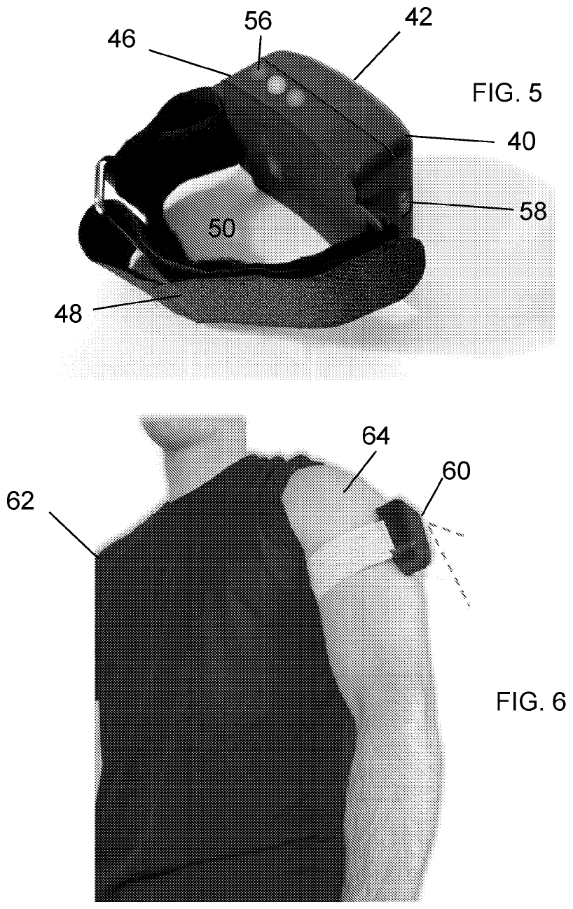

Another example of an embodiment of a wearable smart device 40 is shown in FIGS. 4 and 5. The device 40 includes a cover 42 of a housing 44 that carries an interchangeable battery. A base 46 of the housing 44 is connected to an arm band or strap 48. The base 46 of the housing contains sensors 50 as discussed above and an antidote auto-injector mechanism 52. Control electronics 54 of the sensing system are contained in the housing and control operation of the device 40. The base 46 of the housing also includes operational status indicating LEDs or lights 56 and an override switch 58.

The auto-injector mechanism provided in the wearable device is responsible for dispensing naloxone or like antidote and is designed for intramuscular (IM) or subcutaneous injection. According to some embodiments, the auto-injector mechanism is secured and maintained on the shoulder/upper arm region of the wearer for intramuscular administration of the antidote to the shoulder area of the wearer.

As disclosed above, the housing of the wearable device may contain some or all components that comprise the device. A strap or like securement or stabilization means (i.e., adhesive, arm strap, shoulder strap, etc.) may be used to connect the housing to or adjacent to the shoulder of the wearer so that any injection is an intramuscular injection and occurs within a substantial portion of a muscle, such as in the deltoid muscle of the shoulder of the wearer. Other placements of the device are also contemplated for intramuscular injection.

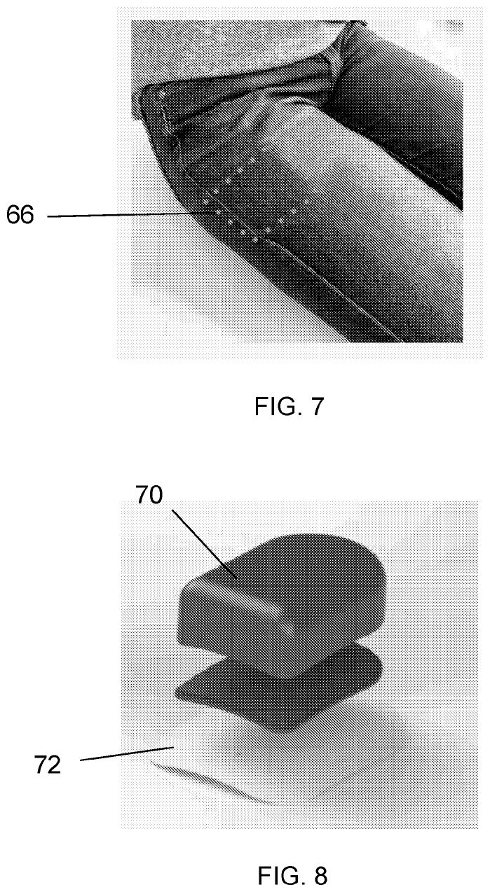

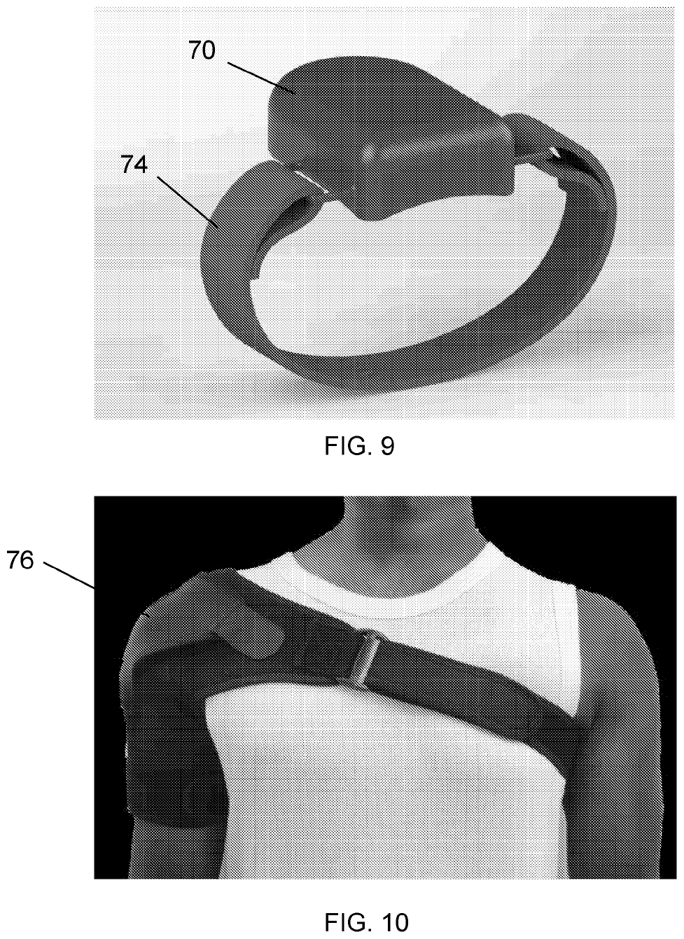

The stabilization system maintains the placement of the device on the wearer and may include one or more of an adhesive and an arm or shoulder strap configuration. By way of example and not by way of limitation, FIG. 6 shows a device 60 worn on the upper arm of a wearer 62 adjacent the wearer's shoulder 64. FIG. 7 shows a potential location 66 on an upper thigh of a wearer. FIG. 8 shows a device 70 for use with an adhesive pad 72 to secure the device 70 to the skin of the wearer. FIG. 9 shows the device 70 provided with a securement strap 74, and FIG. 10 shows a shoulder harness or strap 76 that can be used to secure and stabilize a device on the shoulder area of a wearer.

Thus, according to at least some embodiments, the device is worn on the upper arm/shoulder area and may be designed with a form factor such that the device fits discreetly under clothing. The straps of the device may be adjustable to position the device for injection within a substantial portion of a muscle, even on a wearer that is lean. By way of example and not by way of limitation, the wearable smart device may have dimensions of 3″×2″×1″ (70 mm×50 mm×25 mm), for instance, similar to the size of a pack of cigarettes. A fully automated injection mechanism may be capable of injecting about 2 mL of antidote into the IM site when triggered. Average muscular injection depth may be about 6.25 mm. The wearable device may be battery powered enabling the device to be worn for an extended length of time.

Other contemplated embodiments of wearable devices may not include a strap. For instance, the wearable device may be fashioned as a glove or the like having a pulse oximeter for sensing blood oxygenation level and an overdose condition and may transmit a signal to a separate component mounted adjacent the shoulder for triggering antidote delivery. Alternatively, the glove may be used alone solely for overdose detection and for alerting purposes (i.e., without auto-injection of an antidote).

Initially, naloxone or like antidote may be transferred to or loaded within the device via a vial transfer apparatus or the like within a cleanroom so that the antidote may be deployed within the device. According to at least some embodiments, an auto-injector mechanism is provided within the housing of the device and provides the function of dispensing naloxone or like antidote and injecting the antidote intramuscularly to the wearer of the device.

According to some embodiments, a first stage of actuation of drug delivery involves piercing a cartridge containing a predetermined amount of naloxone or like antidote with a needle such as with the use of a loaded spring, and a second stage releases another spring or the like that pushes a plunger to dispense the naloxone or like antidote through a sealed fluid path via a patient needle into the intramuscular injection site of the wearer of the device. Of course, other mechanisms may be used.

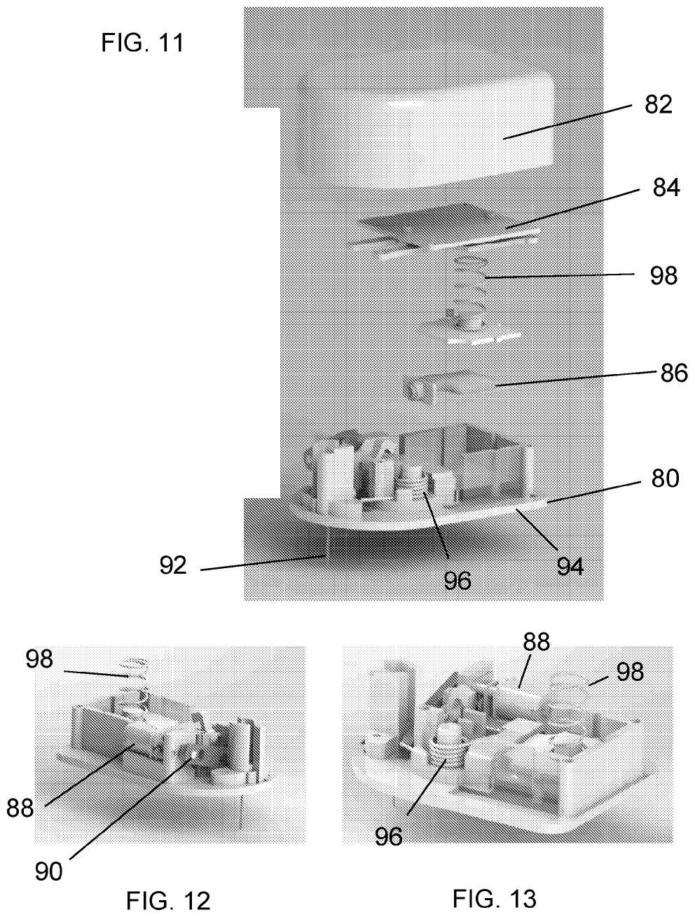

FIGS. 11-13 provide another example of a wearable device 80. The device 80 includes a housing cover 82 in which control electronics 84 and a sealed flexible container 86 of an antidote is located. When an overdose event is detected and automatic injection of an antidote is desired, a small motor 88 is powered to cause rotation of an actuation arm 90. This causes a needle 92 to be extended from a housing base 94 and into the wearer and causes a spring 96 to be released to cause the container 86 to be pierced. The pressure exerted from a second spring 98 can be used to force the antidote out of the pierced container 86 and through the needle 92 and into the wearer.

According to at least some embodiments, the sensor system may be or include a pulse oximeter or like component, i.e., a noninvasive medical device that utilizes spectrophotometry to measure the oxygen saturation of circulating arterial blood in an individual by determining the percentage of oxygenated hemoglobin pulsating through a network of blood capillaries. Thus, the sensor monitors oxygen saturation (SpO₂) and upon sensing an oxygen saturation level below a preset threshold for a predetermined amount of time, may trigger a response to the sensed overdose condition provided that the device also senses that the user is motionless. For this purpose, the sensing system may also include a motion sensor such as an accelerometer or the like to determine whether the wearer of the device is in motion or is motionless (such as during an overdose).

The pulse oximeter sensor of the above referenced embodiment may be modified and calibrated against traditional finger-based pulse oximeter sensors so that accurate measurements can be obtained at the shoulder location or other location of the wearer. For example, the sensor of the wearable device may provide the ability of detecting SpO₂ values in an accurate manner at the shoulder/upper arm location. This enables the sensor and the injector mechanism, both located at the shoulder, to be mounted and contained within the same housing that can be positioned solely on the shoulder/upper arm.

The oximetry sensor may include red and infrared LED emitters and a photodiode used in a reflective mode (i.e., the photodiode receives light reflected from the body of the wearer). The red and infrared LED emitters may have varied or modified intensities relative to conventional pulse oximeters to counter for additional skin, fat, muscle, and various amounts of melanin at the shoulder in comparison to a finger or the like to which conventional pulse oximeters are secured. An example of a sensor calibration operation is provided below.

As discussed above, the wearable device detects critical biomarkers that occur during an opioid overdose and automatically injects naloxone or like antidote, should an overdose be detected. Accordingly, the wearable device accurately detects SpO₂ values off the Opioid Use Disorder (OUD) patient (i.e., wearer of the wearable device). The sensor and auto-injector are housed in a single unit that may be fastened to the shoulder region using a strap mechanism.

The conventional site used to detect pulse oximetry is the finger. Detecting SpO₂ off the shoulder is more difficult due to the following factors: 1) Shoulder composition (Skin, muscle, and fat)-Various OUD patients have different body composition resulting in different perfusion rates, 2) Skin tone (Melanin content)-It is harder to accurately detect SpO₂ levels on patients with higher melanin concentration if the LEDs are not calibrated appropriately; and 3) Pressure application on the sensor against the skin-since there is a wearable arm band, the amount of pressure being applied on the skin by the sensor can vary.

To overcome these hurdles, the wearable device according to an embodiment monitors the sensor values and calibrates the light intensity of the LEDs accordingly. The oximetry sensor may be, for example, a commercially available sensor (MAX30101) provided by Maxim Integrated. The overall functionality of the wearable device is based on the fact that during an opioid-based overdose the individual would be motionless while having respiratory depression which in turn would lead to a drop in SpO₂ values.

Upon starting the wearable device, the device runs a motion sensing algorithm to detect if the user is stationary in a resting phase or exhibiting some form of motion. If the user is motionless, only then will the pulse oximetry LEDs be initiated to start detecting Heart Rate, Respiration rate, and SpO₂%.

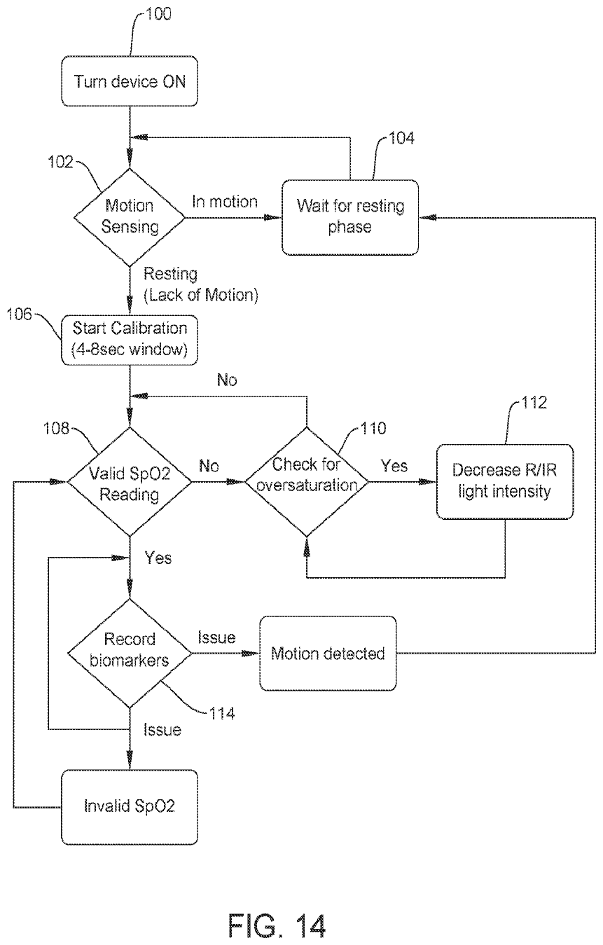

By way of example, FIG. 14 shows operation of the sensors of the wearable device. For instance, the wearable device is turned on in step 100 then the motion sensor begins to sense for motion of the wearer in step 102. If motion of the wearer is detected, then the oximetry sensors remain in an off condition and wait for a resting phase in step 104. When no motion is detected (i.e., a resting phase), calibration of the oximetry sensors is initiated in step 106. A SpO₂ reading is taken and a determination as whether or not such a reading is valid is accomplished in step 108. If the reading is considered invalid, an oversaturation condition is checked in step 110. If an oversaturation condition is detected, then Red/Infrared light intensity is decreased in step 112 and oversaturation is checked again. These steps are repeated until valid SpO₂ readings are detected. Valid readings are recorded in step 114, and this continues until an invalid reading is detected or if motion is detected.

The LED calibration phase depends on the following factors: 1) LED Light Intensity; 2) Pulse width; 3) Samples per second; 4) Sample average; and 5) Analogue to Digital Converter (ADC) count resolution. With respect to LED light intensity, varying the individual light intensities of the Red (650 nm to 670 nm wavelength) and InfraRed (870 nm-900 nm wavelength) wavelengths are crucial to the calibration step. The input current for the LEDs determines the light intensity, i.e., Red (650 nm to 670 nm wavelength)-9 mA to 50 mA and InfraRed (870 nm-900 nm wavelength)-9 mA to 20 mA. With respect to pulse width, individual LED pulse width may be 215 μs to 411 μs. With respect to samples per second, 200-400 samples per second may be recorded for each LED in First In First Out (FIFO) order. With respect to sample average, 4-8 samples may be averaged together. With respect to ADC count resolution, 18 bit ADC resolution, Range [0-262143], may be used.

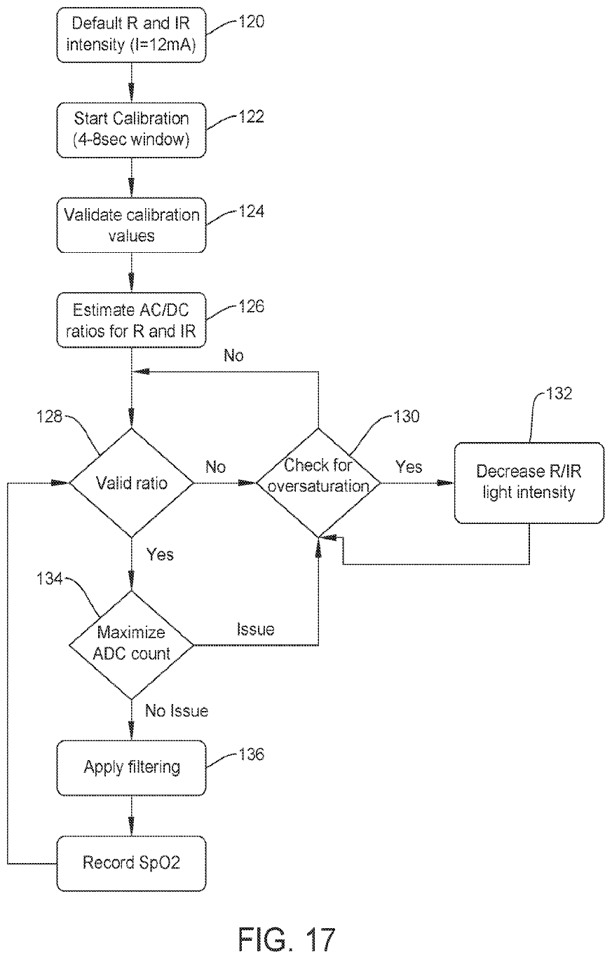

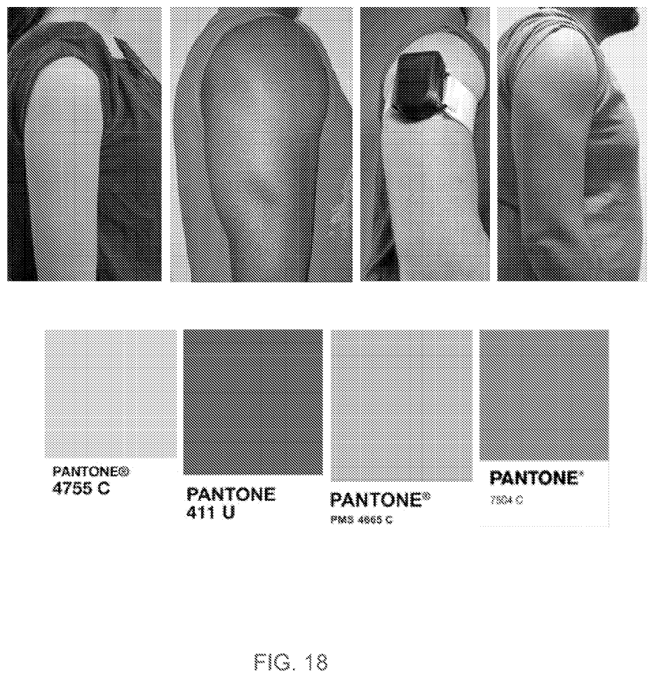

The calibration process for determining light intensities may be as follows and as shown in FIG. 17. Default initial current values of 12 mA are set for both the Red and InfraRed LEDs (see step 120). Then, 4-8 seconds worth of ADC count values from the LED are recorded to calibrate the sensors based on factors mentioned above (see steps 122 and 124). Thereafter, an AC/DC ratio (R) is calculated in step 126 based on the following formula:

$$R = \frac{A {C}_{red} / D {C}_{red}}{A {C}_{ired} / D {C}_{ired}}$$

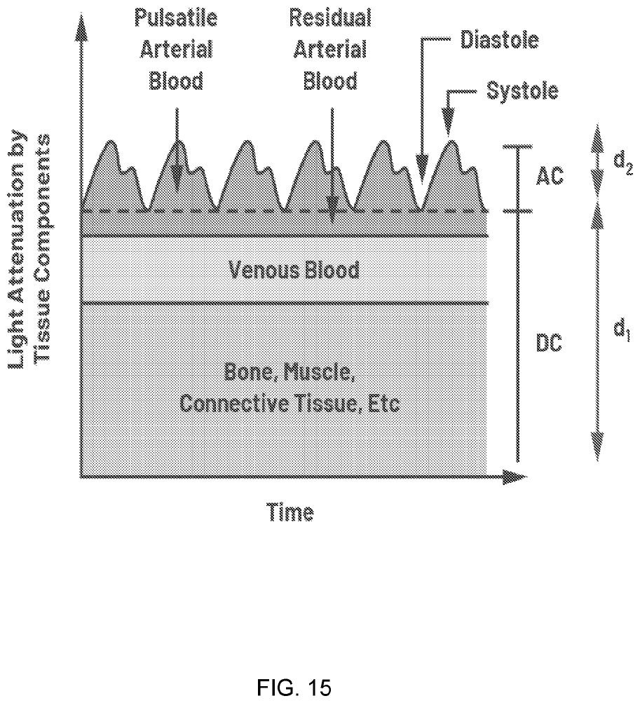

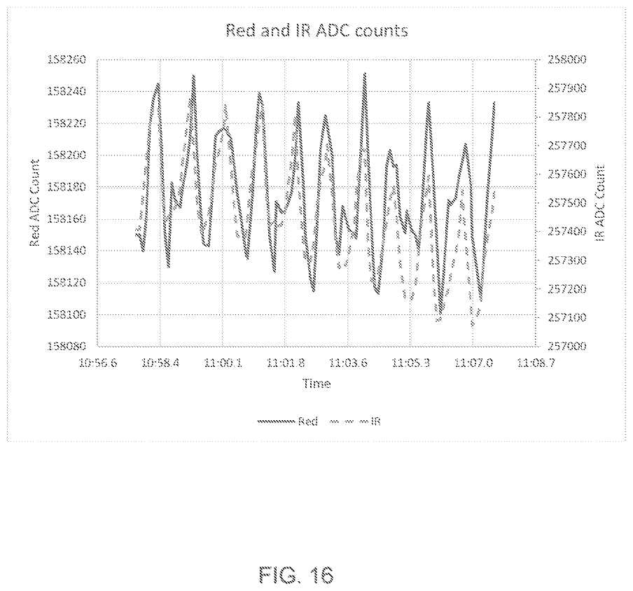

FIG. 15 shows an example of light attenuation by tissue components. The pulsatile arterial blood (during diastole and systole) is shown as the AC component. The residual arterial blood, venous blood, and bone, muscle, connective tissues, etc. are shown as the DC component. FIG. 16 shows an example of Red ADC count and IR ADC count over time.

The R ratio range (i.e., 0.02 to 1.84) is checked in step 128. If the R ratio is invalid, the R and IR counts are checked for oversaturation in step 130. If so, the light intensity is decreased for each LED respectively by a factor of 2 mA (see step 132) and ratio R is recalculated. If the R ratio is within the limit, the ADC count is checked in step 134 to see if it is close to the maximum resolution 18 bits (262143). If not, current input is increased for the LED and the ratio R is recalculated. If R ratio and ADC count are at optimum values, then signal processing is applied in step 136 to improve SpO₂ accuracy at lower blood oxygen levels. The DC component as mentioned above may be removed (Mean-centering) and the values are centered around 0. The mean-centered sample signal is normalized to have a horizontal trendline, and correlation coefficient-based filtering of motion noise in the Red signal is accomplished by using the IR signal as the base model. The Red LED is more susceptible to motion artifacts.

The above referenced calibration system was evaluated by a lab experiment consisting of N=4 participants, each consisting of a different body type and skin tone. The wearable device was assessed on each individual five times to validate the calibration step of setting the correct light intensities and also validating it against a commercial FDA approved pulse oximeter Examples of four body composition types and skin tones measured using the Pantone scale for the validation step are shown in FIG. 18.

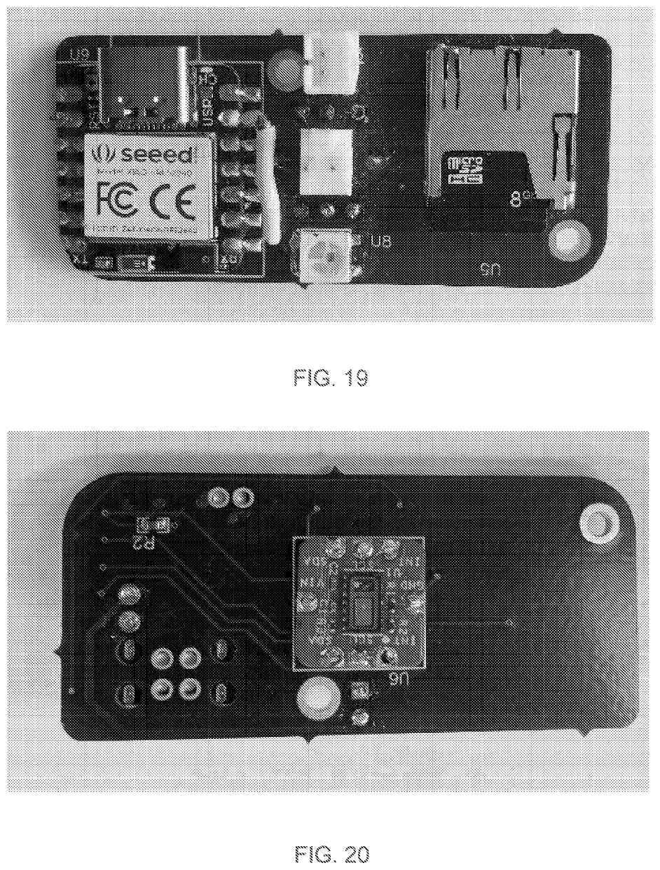

After calibration of the sensors, the sensors were validated against a commercial FDA approved pulse oximeter, the wearable device demonstrated a low error rate of 1%-2% off the commercial pulse oximeter between the four participants. The four participants were healthy volunteers with no prior health conditions resulting in the SpO₂ levels to be between 96%-100%. The top and bottom of the actual sensor used in the above experiment are shown in FIGS. 19 and 20.

Thus, embodiments of the wearable device may monitor the wearer's peripheral oxygen saturation (SpO₂) and movement directly following opioid use and are able to automatically recognize a life-threatening opioid overdose. Upon such recognition, the device may first activate an alarm to notify the wearer or a bystander that an overdose condition has been detected. If the wearer and/or a bystander, if any, does not respond to the alarm and activate an override switch, the device may automatically administer intramuscular (IM) naloxone or like antidote, transmit a signal to first responders (voice, text, etc.), and/or notify community members via a message sent or posted to a mobile app or the like. The wearable device includes an override button or switch to allow the wearer to stop an inadvertent unnecessary injection.

At least some embodiments of the wearable devices may address issues concerning wake-up of a wearer having been injected with an antidote in attempt to make the wake-up period more pleasant (i.e., reduce nausea, vomiting, and like conditions typically experienced upon treatment with naloxone).

According to one embodiment, the wearable device may operate to provide titrated delivery of the antidote (i.e., delivery of the antidote successively in small increments that may be ceased at any time when no longer necessary). For instance, the auto-injector mechanism may be set to deliver only a fraction or part of a supply of the antidote carried within the device. Thereafter, further readings from the sensors may obtained and analyzed. For instance, if the sensors detect that the oxygen saturation level is rising to an acceptable level or if the accelerometer detects movement of the wearer, no further antidote may be delivered by the auto-injector mechanism thereby limiting the overall amount of antidote injected into the wearer. Alternatively, if such readings are not detected by the sensors, the auto-injector mechanism may be activated to deliver a further fraction or part of the supply of antidote carried within the device. This procedure may be continued for several iterations until the full supply has been delivered. Thus, according to this embodiment, only a needed amount of antidote is delivered and not an excess amount (i.e., delivery is halted when overdose reversal is detected).

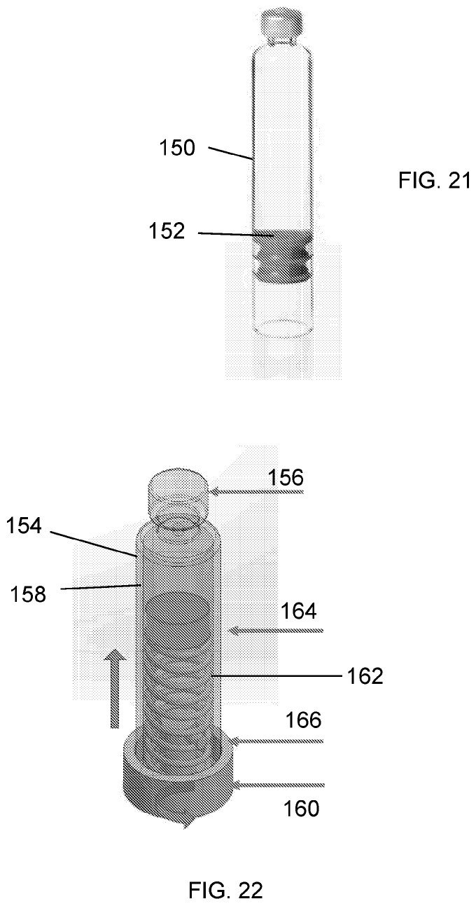

With respect to such a physiology-based titration mechanism for delivering naloxone or like antidote, the required amount of the antidote may be stored in a vial 150, such as a SG Nexa 3X vial manufactured by Strevanato Group, as shown in FIG. 21. The amount of the antidote is then titrated based on physiological changes sensed by the wearable device. For instance, a plunger 152 or the like could be caused to advance within the vial 150 to deliver a predetermined fraction of the antidote.

According to one contemplated embodiment, the amount of antidote that is delivered is controlled by a motor and spring mechanism 154 as shown in FIG. 22. A container side needle may poke through a rubber stopper 156 to create a fluid path between the vial 158 and the patient side needle. As a motor rotates a spring holder 160 at the base of the vial 158, a compressed spring 162 is permitted to slowly decompress as it is extended beyond a flow control notch 166 to linearly push a plunger 164 toward the stopper 156. This in turn pushes the drug/antidote through the needle via the fluid path into the patient. Thus, causing the motor to turn on and off controls the amount of antidote injected into the wearer of the wearable device and can be repeatedly started, stopped, and restarted.

As a further alternative, the antidote carried by the wearable device may be a single substance, such as naloxone, or may be a mixture of substances that may allow for a more pleasant wake up of the wearer having been injected with the antidote. For example, with respect to a mixture, the antidote may consist of an amount of naloxone mixed with an antiemetic drug that may prevent, reduce, or treat nausea, vomiting, and like conditions caused by antidote drug treatment. As an example of an antiemetic drug, ondansetron or the like may be used. In addition, the antidote may contain an opioid antagonist other than naloxone, or a mixture of different opioid antagonists, such as naloxone mixed with buprenorphine or the like.

While the invention has been described with reference to particular embodiments, it will be appreciated that modifications can be made without departing from the spirit of the invention. Such modifications are intended to fall within the scope of the appended claims.

What is claimed is:

1. A wearable device (10, 40, 60, 70, 80) for recognition and response to an overdose, comprising:
an oximetry sensor (22, 32, 50) for measuring peripheral oxygen saturation (SpO₂) of a wearer of the wearable device (10, 40, 60, 70, 80); and
a motion sensor (22, 34, 50) for determining whether the wearer of the wearable device (10, 40, 60, 70, 80) is moving or motionless.

2. The wearable device (10, 40, 60, 70, 80) according to claim 1, further comprising an injector (52) including a supply (18, 86) of an antidote and a needle (20, 92) for intramuscular or subcutaneous delivery of the antidote to the wearer of the wearable device (10, 40, 60, 70, 80).

3. The wearable device (10, 40, 60, 70, 80) according to claim 2, further comprising a housing (14, 44, 82, 94) in which the oximetry sensor (32), the motion sensor (34), and the supply (18, 86) of the antidote is contained and in which the needle (20, 92) is initially contained in a retracted condition.

4. The wearable device (10, 40, 60, 70, 80) according to claim 3, further comprising a strap (16, 48, 74, 76) extending from the housing (14, 44) and sized to secure the housing (14, 44) on an upper arm of the wearer adjacent the shoulder of the wearer.

5. The wearable device (10, 40, 60, 70, 80) according to claim 3, further comprising control electronics (24, 54, 84) housed within said housing (14, 44, 82, 94) and being configured to receive and analyze readings of the oximetry sensor (32) and the motion sensor (34) and to detect a suspected overdose from said readings.

6. The wearable device (10, 40, 60, 70, 80) according to claim 5, wherein said control electronics (24, 54, 84) are configured to determine a suspected overdose when peripheral oxygen saturation (SpO₂) measured by said oximetry sensor (32) falls below a predetermined oxygen saturation threshold level and said motion sensor (34) indicates that the wearer of the wearable device (10, 40, 60, 70, 80) is motionless.

7. The wearable device (10, 40, 60, 70, 80) according to claim 6, when said control electronics (24, 54, 84) are configured to cause an audible or vibratory alarm to be emitted by the wearable device (10, 40, 60, 70, 80) upon determination of a suspected overdose.

8. The wearable device (10, 40, 60, 70, 80) according to claim 7, further comprising an override button or switch (28, 58) carried by the housing (14, 44), wherein said control electronics (24, 54, 84) are configured to actuate said injector when the override button or switch (28, 58) is not actuated within a predetermined period of time following a commencement of the alarm and if the motion sensor (34) continues to indicate that the wearer of the device (10, 40, 60, 70, 80) is motionless.

9. The wearable device (10, 40, 60, 70, 80) according to claim 8, wherein, when said injector (52) is actuated by said control electronics (24, 54, 84), said needle (20, 92) is extended from said housing (14, 44, 82, 94) in a direction to pierce the skin and muscle of the wearer and at least a portion of said supply of antidote is forced through said needle (20, 92) for intramuscular injection of the antidote to the wearer of the wearable device (10, 40, 60, 70, 80).

10. The wearable device (80) according to claim 9, wherein the injector includes a motor (88) for rotating an actuation arm (90) configured to extend the needle (92).

11. The wearable device (80) according to claim 10, wherein the injector includes a spring (96) for causing piercing of a vessel (86) within the housing (82, 94) containing the supply of antidote.

12. The wearable device (80) according to claim 11, wherein the vessel (86) is flexible, and wherein injector includes a second spring (98) for forcing the supply of antidote out of the vessel (86) when the vessel (86) is pierced.

13. The wearable device (10, 40, 60, 70, 80) according to claim 8, wherein, when said injector is actuated by said control electronics (24, 54, 84), the ejector is caused to inject a partial portion of the supply of antidote and to thereafter analyze additional readings from the oximetry sensor (32) and motion sensor (34) to determine if motion of the wearer is indicated by the motion sensor (34) or if peripheral oxygen saturation (SpO₂) measured by said oximetry sensor (32) rises to a level above the predetermined oxygen saturation threshold level.

14. The wearable device (10, 40, 60, 70, 80) according to claim 13, wherein said injector is caused to cease further injection of the antidote if motion of the wearer is indicated by the motion sensor (34) or if peripheral oxygen saturation (SpO₂) measured by said oximetry sensor (32) rises to a level above the predetermined oxygen saturation threshold level.

15. The wearable device (10, 40, 60, 70, 80) according to claim 14, wherein said control electronics (24, 54, 84) are configured to cause the injector to inject an additional portion of the supply of antidote after the additional readings are analyzed if no motion of the wearer is indicated by the motion sensor (34) and if peripheral oxygen saturation (SpO₂) measured by said oximetry sensor (32) remains below the predetermined oxygen saturation threshold level.

16. The wearable device (10, 40, 60, 70, 80) according to claim 6, wherein the control electronics (24, 54, 84) include a transmitter, and wherein the control electronics (24, 54, 84) are configured to cause said transmitter to initiate transmission of a message or signal to a responder or community member upon detection of a suspected overdose, and wherein a battery (26) contained with said housing (14, 44, 82, 94) for providing a power source for said control electronics (24, 54, 84).

17. The wearable device (10, 40, 60, 70, 80) according to claim 2, wherein the antidote comprises an opioid antagonist, naloxone, or naloxone mixed with another opioid antagonist.

18. The wearable device (10, 40, 60, 70, 80) according to claim 17, wherein the antidote further comprises an antiemetic.

19. The wearable device (10, 40, 60, 70, 80) according to claim 1, wherein said oximetry sensor (32) operates in a reflective mode and includes red and infrared light emitting diodes (LEDs) and a photodiode, wherein said red LED has a light intensity within a range of 650 nm to 670 nm wavelength and said infrared LED has a light intensity within a range of 870 nm to 900 nm wavelength, and wherein a ratio of the light intensity of said red LED to the light intensity of said infrared LED is within a range of 0.02 to 1.84.

20. The wearable device (10, 40, 60, 70, 80) according to claim 1, wherein the motion sensor (34) is an accelerometer.
